(19) United States
(12) Patent Application Publication
(10) Pub. No.: US 2015/0257748 A1
(43) Pub. Date: Sep. 17, 2015

(54) LAPAROSCOPIC DEVICE CONFIGURED WITH ERGONOMIC CONTROL FOR EXTERNALLY MANIPULATING AN INTERNAL ORGRAN

(71) Applicant: Rasiklal Shamji Shah, Mumbai (IN)

(72) Inventor: Rasiklal Shamji Shah, Mumbai (IN)

(21) Appl. No.: 14/436,885

(22) PCT Filed: Apr. 4, 2013
(86) PCT No.: PCT/IN2013/000226

§ 371 Date: Apr. 18, 2015

(30) Foreign Application Priority Data
Dec. 31, 2012 (IN) 3728/MUM/2012

(51) Int. Cl. A61B 17/04; A61B 17/02

(52) U.S. Cl. CPC A61B 17/0401; A61B 17/0483; A61B 17/0493; A61B 17/0218; A61B 2017/00876

(57) ABSTRACT

A laparoscopic device configured with an ergonomic external control to manipulate an internal organ, the laparoscopic device comprises: an introducer sheath having an open inner end and an open outer end fitted with an annular disc; the opening in the annular disc adapted to be closed in a sealing manner by means of a washer; at least one anchor attached to a common flexible string which is subsequently divided into a plurality of flexible strings extending out of the introducer sheath through the opening in the washer; the anchor being disposed inside the introducer sheath; means for securing the anchor inside the introducer sheath and means for releasing the anchor into the abdomen; wherein the plurality of flexible strings passing through the opening in the washer are adapted to be fixed in a pivotable clip and each of said plurality of strings passes through a skin guard having a string director for externally manipulating the internal organ with ergonomic control.

### FIELD OF THE INVENTION

The present invention relates to a laparoscopic device configured with ergonomic external control to manipulate an internal organ, using an anchor, which is attached to strings that exits from the abdominal wall at selected locations.

### BACKGROUND OF THE INVENTION

The art of laparoscopy has been known to the mankind for more than 100 years. However, the rapid growth has occurred in this field only after 1986, when the first laparoscopic cholecystectomy was reported by a surgeon from France.

Laparoscopic surgery is a form of surgery carried out through small incisions made on the abdominal wall, instead of making conventional large incisions. This surgery requires the use of special tubes (called trocars), hand instruments, telescope and camera. Laparoscopic surgery is performed by making small incisions on the abdominal wall and then inserting several trocars through these incisions. Thereafter, a telescope is inserted through one of these trocars to observe inside view of the abdominal cavity. Subsequently, laparoscopic hand instruments are introduced through these trocars for manipulating abdominal viscera to perform different activities, such as-retraction, dissection, cutting, suturing etc. to carry out laparoscopic surgery.

At present, many laparoscopic surgeries are being performed using this technique. One of the most common surgeries being performed by this technique is cholecystectomy (i.e. removal of the gallbladder). Normally, the laparoscopic cholecystectomy is performed using four trocars. The first trocar is inserted at the umbilicus and the abdomen is filled up with carbon dioxide gas pumped in with the help of a pump (called the insufflator). This creates the space required in the abdomen for the ease of the surgeon to work inside the abdomen. Through this trocar, a telescope is passed which is connected to a small camera (called endocamera). This telescope and camera pick-up internal image of the abdominal cavity and display it on a monitor. Three more trocars are inserted in the abdomen for introducing various laparoscopic hand instruments that enable the surgeon to carry out various surgical steps. The position of these trocars varies from surgeon to surgeon and also from patient to patient.

First step of the laparoscopic cholecystectomy is to grasp the fundus (upper part) of the gallbladder and push it towards the diaphragm with the use of a grasping forceps from a trocar on the right side of the abdomen. Alternatively, the laparoscopic device as per the present inventor's earlier Indian patent application no. 3012/MUM/2012 may be used to achieve a similar result with the associated benefits as per the details of that patent application. This step exposes an area called “Calot's triangle” which carries the artery and the duct entering the gallbladder (cystic artery and cystic duct).

The second step is to manipulate the Hartman's Pouch of the gallbladder, so that the medial and lateral part of the gall bladder can be seen. In a standard laparoscopic cholecystectomy, this step is normally carried out by the grasper placed through a trocar in the midclavicular line. Then, either a hook or a dissecting forceps is introduced through the epigastric trocar for dissecting the cystic artery and cystic duct.

The surgeon manipulates Hartman's pouch of gallbladder with left hand using a grasper and dissects with the L Shape diathermy hook or dissecting forceps with the right hand. During this step of surgery the shoulder joints of the surgeon remain in an abducted position; which is also known as “Chicken Wing Scapula”. This abducted position of the shoulder joints of the laparoscopic surgeon substantially increases the wear and tear of these joints, which in some surgeons even necessitates a shoulder surgery. Abduction of the shoulder joints up to 30 degree has almost no stress and it can be achieved by using the device according to this invention.

A recent modification to the technique of laparoscopic cholecystectomy has been the single incision laparoscopic cholecystectomy. As the name itself suggests, only one incision is made near the umbilicus in this form of surgery, and all the instruments as well as the telescope are introduced through various trocars placed side by side or through a specialized device that has up to three or four channels. Here, one of the instruments gets utilized in retraction of the fundus of the gallbladder towards the thoracic diaphragm and others to manipulate Hartman's pouch to safely complete the procedure.

In a single incision laparoscopic surgery, the manipulation of Hartman's pouch is carried out by the instrument, which is inserted from one of the trocar in multiport technique or from the opening of a special trocar in single port surgery. By using an anchor attached to multiple strings, as described in accordance with this invention, it is possible to reduce at least one instrument that needs to be introduced though the umbilicus. This will also increase the freedom of the movement of the remaining instruments. The ergonomic external control allows the surgeon to work with the shoulder joints in the natural position, which is likely to further decrease the stress level and discomfort to the operating surgeon, which ultimately will result in a safer laparoscopic surgery.

Similarly, laparoscopic excision of the fibroid of the uterus or any other benign solid tumor is performed using three or four trocars. The surgeon manipulates the fibroid of the uterus or benign solid tumor by using a myoma screw or a grasper. By use of the presently disclosed Screw Anchor attached to the multiple strings, it is possible to reduce one trocar and an instrument that is needed to manipulate the fibroid of the uterus or benign solid tumor. The ergonomic external control of these strings is likely to reduce the wear and tear of the shoulder joints of the surgeon, simplify the surgery and is also likely to produce better results.

In case of the surgery at the esophageal hiatus, multiple trocars are placed in the upper abdomen. One of the main step of surgery at the esophageal hiatus is to retract the left lobe of the liver and at present many laparoscopic retractors are available to carry out this step. However, the main problem with these retractors is that they move during the surgery and they need to be often repositioned during the surgery. In addition, a trocar and an assistant are required to hold this liver retractor in position.

To manipulate small or large bowel, graspers are used which also have a similar problem of wide abduction at the shoulder joints of the operating surgeon. The anchor clip attached to the multiple strings in accordance with the present invention provides the surgeon an ergonomically superior position and comfortable option to manipulate the internal organ in multiple directions.

### OBJECTS OF THE INVENTION

Some of the objects of the present disclosure, which at least one embodiment of the present invention satisfies, are as follows:

It is an object of the present invention to provide a laparoscopic device, which reduces the number of trocars used in laparoscopic surgery and thus reducing the cost of surgery.

It is another object of the present invention to provide a laparoscopic device, which offers the laparoscopic surgeon an ergonomically superior position, specifically to reduce the abduction at the shoulder joints of the surgeon.

It is still another object of the present invention to provide a laparoscopic device, which reduces the stress to the shoulder joints of the surgeon and associated wear and tear thereof.

It is a further object of the present invention to provide a laparoscopic device, which decreases the number of instruments that need to be inserted from the umbilicus during a single incision laparoscopic surgery.

It is a yet another object of the present invention to provide a laparoscopic device, which increases the freedom of movement of the instruments that are inserted through the umbilicus and to facilitate the ease with which single incision laparoscopic surgery can be performed by the laparoscopic surgeon.

It is a still further object of the present invention to provide a laparoscopic device, which simplifies the laparoscopic procedure by enabling a good retraction and satisfactory exposure of Calot's triangle during a single incision laparoscopic cholecystectomy.

It is a yet further object of the present invention to provide a laparoscopic device, which enables a good retraction of Hartman's pouch of the gallbladder, allowing safe dissection of Calot's triangle, thereby aiding to reduce the chances of injury to the bile ducts (tubes that carry bile from the liver to the intestine).

It is also an object of the present invention to provide a laparoscopic device, which dispenses with the need to introduce the myoma screw and the need of an assistant in laparoscopic excision of fibroid of the uterus.

It is a further object of the present invention to provide a laparoscopic device, which allows easy manipulation and retraction of the uterine fibroid and benign solid tumors during their laparoscopic surgical excision.

It is yet further object of the present invention to provide a laparoscopic device, which enables a good retraction of the left lobe of the liver during laparoscopic surgeries that are performed at the esophageal hiatus.

Other objects and advantages of the present invention will be more apparent from the following description when read in conjunction with the accompanying figures of drawings, which are however not intended to limit the scope of the present invention in any way.

### SUMMARY OF THE INVENTION

In accordance with the present invention, there is provided a laparoscopic device configured with ergonomic external control to manipulate an internal organ, the laparoscopic device comprises of:
an introducer sheath having an open inner end and an open outer end fitted with an annular disc;
an opening in the annular disc that is adapted to be closed in a sealing manner by means of a washer;
at least one anchor attached to a common flexible string, which is subsequently divided into a plurality of flexible strings extending out of the introducer sheath through the opening in the washer; the anchor being disposed inside the introducer sheath;
means for securing the anchor inside the introducer sheath and for releasing the anchor into the abdomen;
   wherein the plurality of flexible strings passing through the opening in the washer are adapted to be fixed in a pivotable clip and each of the said plurality of string passes through a skin guard having a string director for manipulating the internal organ with ergonomic external control.

Typically, the anchor is configured as at least partially magnetic T-fastener, which also has a substantially annular flexible disc disposed at the junction of the common flexible string with the T-fastener.

Typically, the means for introducing the T-fastener comprises of an introducer needle having an open, sharp, slotted inner end and an open outer end provided with an annular disc having a locking arrangement.

Typically, the locking arrangement comprises of a pivoting arm extending from the annular disc and having a hook lockable on the annular disc of the introducer sheath to lock the introducer needle in the introducer sheath or to release the introducer needle from the annular disc of the introducer sheath to move the introducer needle out of the introducer sheath.

Typically, the means for securing the T-fastener is a magnet fitted adjacent to the slotted end of the introducer needle.

Typically, the means for releasing the T-fastener out of the introducer needle is T-fastener pusher provided with a circular disc having a locking arrangement which is lockable on the annular disc of the introducer needle to lock the T-fastener pusher inside the introducer needle and to release the T-fastener pusher in order to push the T-fastener out of the introducer needle and thereby to release T-fastener into the internal organ.

Typically, the T-fastener pusher has a concaved profile at its end abutting the T-fastener and the T-fastener has complementary convex profiles at both ends thereof.

Typically, the anchor is configured as a hook anchor having at least one fluke.

Typically, the anchor is configured as a clip anchor.

Typically, the means for securing the hook anchor or clip anchor inside the introducer sheath is a grasper assembly.

Typically, the grasper assembly comprises of the locking means partially disposed outside the introducer sheath and the grasping means disposed inside the introducer sheath, the locking means and grasping means cooperating with each other to grasp or release the hook anchor or clip anchor.

Typically, the anchor comprises of a hook anchor having at least one fluke and shank of the hook anchor is attached with a common flexible string on one side and a piece of thread attached to the opposite side.

Typically, the anchor is a screw anchor.

Typically, the screw anchor having external threads at the rear end is screwed or unscrewed using a screwdriver having a cylindrical arm provided with complementary internal threads at its front end and a handle firmly fitted at its rear end which is disposed outside the introducer sheath.

Typically, the common flexible string of the screw anchor is attached to a non-flexible disc fitted at the rear end of the screw head, the non-flexible disc acts as a stopper while the screw anchor is being screwed into an internal organ, the external screw threads of the screw head provided at its rear end are adapted to be engaged by the internal threads of the handle of the screwdriver.

Typically, the screw head is provided with a plurality of spikes in the anti-clockwise direction for preventing detachment of the screw head from the internal organ.

Typically, the pivotable clip comprises of an upper and lower rectangular portion which are adapted to be pivoted with respect to each other on one side thereof and also adapted to be locked to each other on the other side thereof for fixing the respective string in position within the pivotable clip.

Typically, the pivotable clip comprises of a base provided with an adhesive surface having a removable cover for sticking it on the abdomen.

Typically, the skin guard is configured with an inclined slope to protect the skin by reducing friction at the exit site of the respective string and to retain the string on the skin guard and to direct it towards the pivotable clip.

Typically, the skin guard is configured as a cylindrical body with a through slit at one point on the circumference thereof, the slit extending proximate to the central region of the skin guard and then transforming into a smooth inclined slope ending near its top face which is depressed by a predetermined height to configure a depression from the top surface of the skin guard thereby forming a grooved recess to retain the string on the skin guard.

Typically, the skin guard comprises of a base, which is provided with an adhesive surface having a removable cover for sticking it on the abdomen.

### BRIEF DESCRIPTION OF THE ACCOMPANYING DRAWINGS

The laparoscopic device in accordance with the embodiments of the present invention will now be explained in more detail with reference to the non-limiting accompanying drawings in which:

FIG. 1 illustrates a T-fastener anchor attached with a common string, which is subsequently divided into flexible strings for the laparoscopic device in accordance with the present invention.

FIG. 2 illustrates the laparoscopic device in accordance with the present invention, showing a T-fastener anchor attached with a common string subsequently dividing into flexible strings as shown in FIG. 1, and which is introduced in the slotted introducer needle, both of which in turn are housed together in the introducer sheath along with a T-fastener pusher.

FIG. 3 illustrates an enlarged view of the inner end of the introducer sheath assembly shown in FIG. 2.

FIG. 4 illustrates the external view of the abdomen showing flexible strings (as shown in FIG. 1) exiting from various locations on the abdomen and which are shown along with the respective skin guards and string directors, passing through a pivotable clip.

FIG. 5 illustrates the hook anchor attached with a common string subsequently dividing into flexible strings for the laparoscopic device in accordance with the present invention.

FIG. 6 illustrates the laparoscopic device in accordance with the present invention, showing a hook anchor attached with a common string subsequently dividing into flexible strings (as shown in FIG. 5)—which is grasped by the grasper inside the introducing sheath.

FIG. 7 illustrates a clip anchor attached with a common string subsequently dividing into flexible strings,

FIG. 8 illustrates the laparoscopic device in accordance with the present invention, showing a clip anchor attached with a common string subsequently dividing into flexible strings (as shown in FIG. 7), which is grasped by the grasper inside the introducing sheath.

FIG. 9 illustrates the laparoscopic device in accordance with the present invention, showing a screw anchor attached with common string subsequently dividing into flexible strings, the said screw anchor is to be held by the screwdriver inside the introducing sheath.

FIG. 10 illustrates the washer with the introducing sheath of the laparoscopic device in accordance with the present invention.

FIG. 11 illustrates the hypodermic needle with a suture loop used for pulling flexible strings from inside to outside of the abdomen.

FIG. 12 illustrates different views of the skin guard and string director configured for the laparoscopic device in accordance with the present invention.

FIG. 13 illustrates the pivotable clip for fixing flexible strings on the abdominal wall.

FIG. 14 illustrates cross-sectional view of the abdomen, showing various exit points of the flexible strings which are attached to the common string of the anchor of the laparoscopic device in accordance with the present invention, each of these strings pass through the abdominal wall at the position of the skin guards and respective string directors. It also shows the inside view of the abdomen showing a T-fastener Anchor in the lumen of the gallbladder in the region of Hartman's pouch HP and common string attached to the T-fastener.

### DETAILED DESCRIPTION OF THE ACCOMPANYING DRAWINGS

The laparoscopic device in accordance with the present invention will now be described with reference to the accompanying drawings, which do not limit the scope and ambit of the disclosure. The following description of the exemplary embodiments of the laparoscopic device with an anchor attached to a common string subsequently dividing into multiple flexible strings is provided in order to manipulate an internal organ by an ergonomic external control will so fully reveal the general nature of these embodiments that by applying the current knowledge in the art, others can readily modify and/or adapt these specific embodiments for various applications, without departing from the generic concept of the present invention, and therefore, such adaptations and modifications should be intended and indeed are intended to be comprehended within the meaning and range of the equivalents of the embodiments disclosed in the present specification.

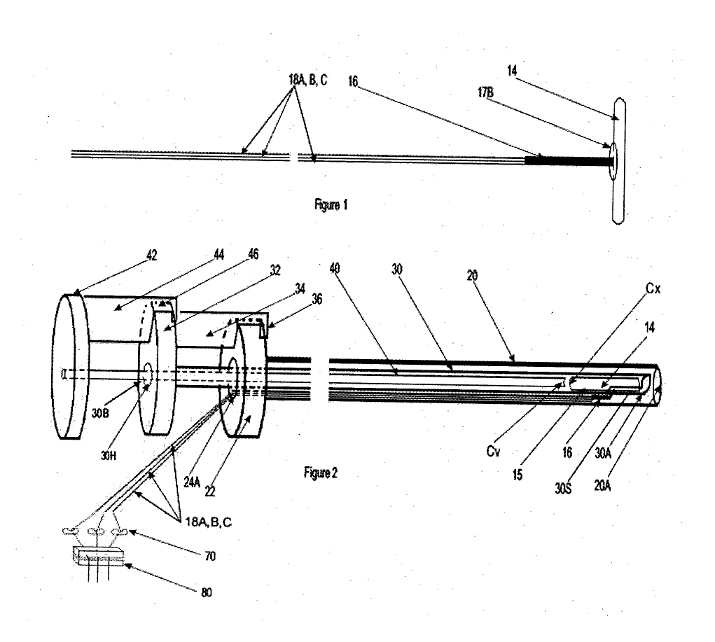

FIG. 1 illustrates a T-fastener anchor 14 attached to a common string 16, which is subsequently divided into multiple flexible strings 18A, 18B and 18C. Although, the FIG. 1 shows the most preferred embodiment of the invention, it is possible to attach the T-fastener 14 directly to the multiple flexible strings 18A, 18B, 18C etc. by omitting the common string 16 shown therein. Alternatively, the common flexible string 16 includes a plurality of multiple flexible strings 18A, 18B, 18C etc. corresponding to each of the multiple flexible strings. The junction of the common string 16 with T-Fastener 14 is fitted with a flexible thin disc 17B, which seals the opening made in the hollow viscera, e.g. in the gallbladder from the inside. The strings 18A, 18B and 18C are given different colour coding for easy identification thereof.

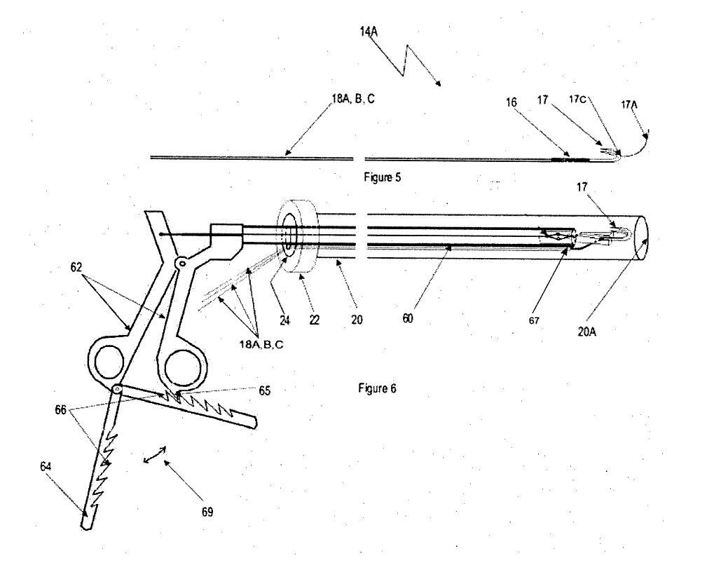

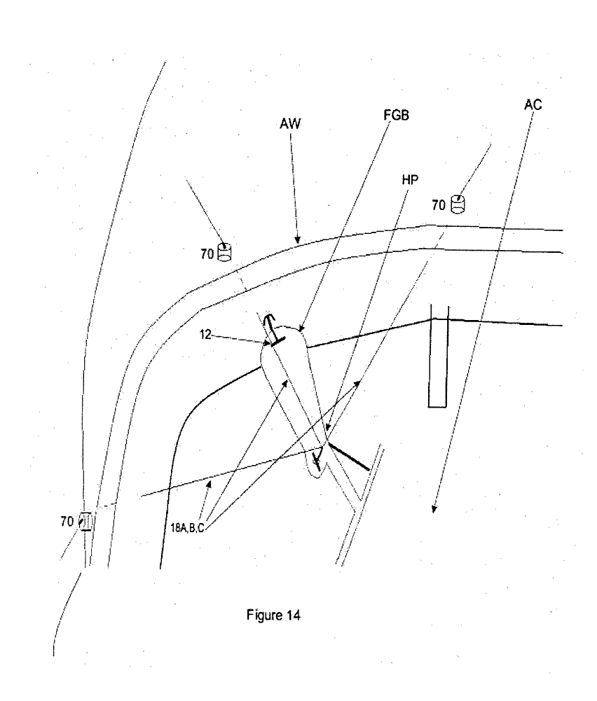

FIG. 2 illustrates the laparoscopic device in accordance with the present invention, in which a T-fastener 14 (shown in FIG. 1) is housed in a slotted introducer needle 30 along with a T-fastener pusher 40, both of which are disposed inside an introducer sheath 20 which is a hollow tubular structure of adequate diameter. The introducer sheath 20 harbors the slotted introducer needle 30, T-fastener pusher 40 and T-Fastener 14 attached to a common string 16 subsequently divided into flexible strings 18A, 18B and 18C as shown in FIG. 1. These strings are provided with the skin guards 70 and are fixed in respective position on the pivotable clip 80. During the use of the laparoscopic device according to this invention, these skin guards 70 and pivotable clip 80 are detached and put in their respective position on the abdomen only after each of these strings 18A, 18B and 18C are brought out of the abdomen as shown in FIGS. 4 and 14. The outer end 20B of the introducer sheath 20 is configured as an annular disc 22. The washer 24 snugly fits over the annual disc 22 and marking 24A depicts opening on both the annular disc 22 and on the washer 24 for the introduction and removal of this slotted introducer needle 30 or the grasper assembly 60 (not shown here). Through the opening 24A in the washer 24 and the annular disc 22, the slotted introducer needle 30 or a grasper assembly 60 is introduced in the introducer sheath 20. The washer 24 snugly grasps the slotted introducer needle 30 or the grasper assembly 60 (not shown here, see FIG. 6). This washer 24 is provided to prevent any leakage of gases from the abdomen via the introducer sheath 20 by the sides of the instruments, which have been introduced through its opening 24A. In a typical laparoscopic cholecystectomy, this laparoscopic device can be used to manipulate Hartman's pouch HP of the gallbladder by manipulating these flexible strings 18A, 18B, and 18C from outside of the abdomen. In addition, these flexible strings 18A, 18B and 18C also exit through this opening 24A, until they are pushed into the abdomen by a grasper assembly 60 (not shown here, see FIG. 6) and are subsequently pulled out of the abdominal wall by a suture loop S (see FIG. 11). The slotted introducer needle 30 along with a T-fastener pusher 40 is also shown here. The long arm of the T-fastener pusher 40 passes through the hole 30H provided at the center of the disc 32 of the slotted introducer needle 30. The slotted introducer needle 30 is a long hollow cylindrical tube similar to a hypodermic needle, which has a sharp inner end 30A for smooth penetration through the Hartman's pouch HP of the gallbladder (see FIG. 14). This sharp end 30A of the needle is provided with a slot 30S for a sidewise passage of the common string 16 through it. Optionally, a magnet 15 fitted near this slot 30S may also form part of this needle-like construction; so that the T-fastener 14 can be secured in its position by the magnetic force created in the slotted introducer needle 30 until it is mechanically pushed out of the slotted introducer needle 30 by the T-fastener pusher 40. A hook 36 of a pivoting arm 34 of the locking arrangement provided on the annular disc 32 of this introducer needle 30 can be locked on the annular disc 22 of the introducer sheath 20. The swiveling movement of the hook 36 in the upwardly direction will release the slotted introducer needle 30, so that it can be pushed inside the introducer sheath 20. Similarly, the T-fastener pusher 40 is a long cylinder, which is also to be introduced through this slotted introducer needle 30. The inner end Cv of the T-fastener pusher 40 facing the T-fastener 14 is configured concave so that it can receive one of the two convex ends Cx of the T-fastener 14. The outer end of the T-fastener pusher 40 also has a circular disc 42 fitted thereon. It also has a similar locking arrangement. The hook 46 provided on the pivoting arm 44 thereof is configured pivotable (not shown) with respect to this circular disc 42. This hook 46 can be locked on the annular disc 32 provided on the outer end 30B of the slotted introducer needle 30. This hook 46 functions in a similar manner to that of the hook 36 of the locking arrangement provided on the outer end of the slotted introducer needle 30. The T-fastener pusher 40 can be similarly released from the annular disc 32 by an upwardly swiveling movement of the pivoting arm 44. Release of this hook 46 allows the inward movement of the T-fastener pusher 40 into the slotted introducer needle 30, which in turn pushes the T-fastener 14 out of the inner end 30A of the slotted introducer needle 30 and thereby delivers it into the lumen of the gallbladder GB (not shown) In the unlocked position, the T-fastener pusher 40 can also be withdrawn completely from the slotted introducer needle 30, so that lumen of the slotted introducer needle 30 has continuity from its outer end 30B to its inner end 30A and thereby it has continuity with the cavity of the gallbladder GB. Now, the outer end 30B of the slotted introducer needle 30 can be connected to a suction tubing to suck out the contents of the gallbladder GB in order to reduce the spillage thereof.

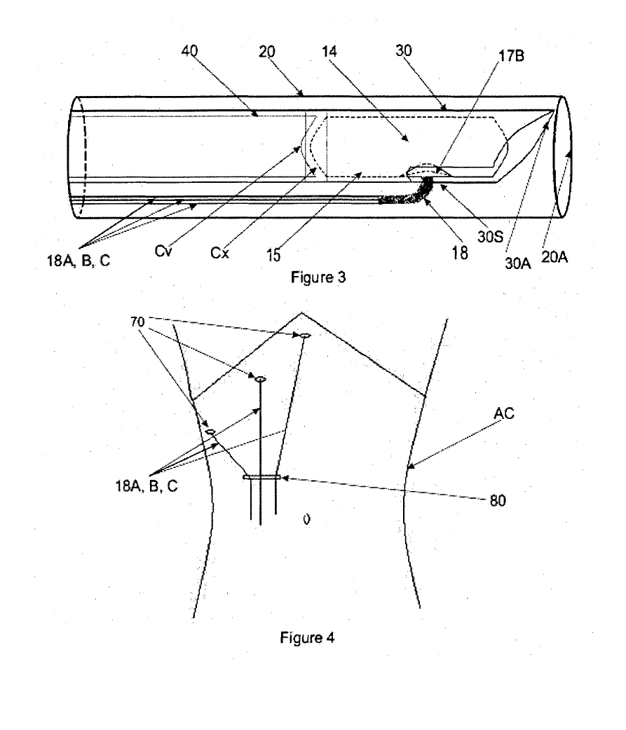

FIG. 3 illustrates an enlarged view at the inner end 20A of the introducer sheath assembly, particularly showing the tip 30A of the slotted introducer needle 30 with the T-fastener 14 housed within the slotted introducer needle 30, which in turn is disposed inside the introducer sheath 20. The complete introducer sheath assembly is inserted into a laparoscopic trocar (not shown) for delivering the present laparoscopic device in the operative region. The complete introducer sheath assembly consists of a T-fastener 14 anchor disposed inside a slotted introducer needle 30, a T-fastener pusher 40, a common string 16 attached to the T-fastener 14. The common string 16 emerges sidewise from the slot 30S of the slotted introducer needle 30, and is subsequently divided into flexible strings 18A, 18B and 18C, which in turn exit from the opening 24A provided at the outer end 20B of the introducer sheath 20 (see FIG. 2).

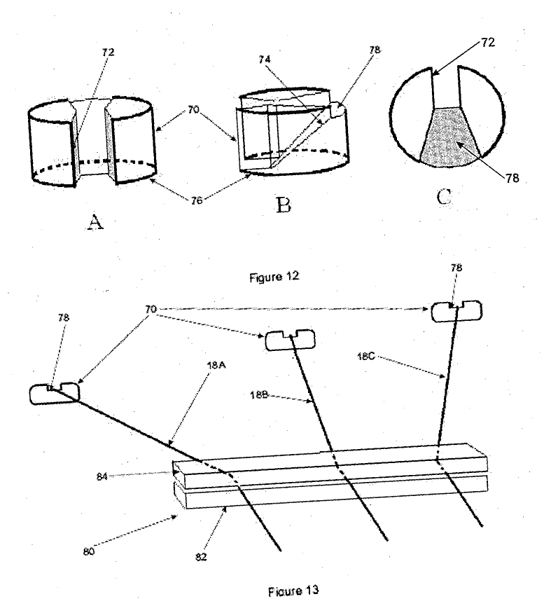

FIG. 4 illustrates an outside view of the abdomen showing flexible strings 18A, 18B and 18C exiting from the abdomen at the specific locations on the abdomen as decided by the surgeon, through the respective skin guards 70 each having a string director 78 (see FIG. 12) and pivotable clip 80 (see FIG. 13 for details) to fix these strings 18A, 18B and 18C.

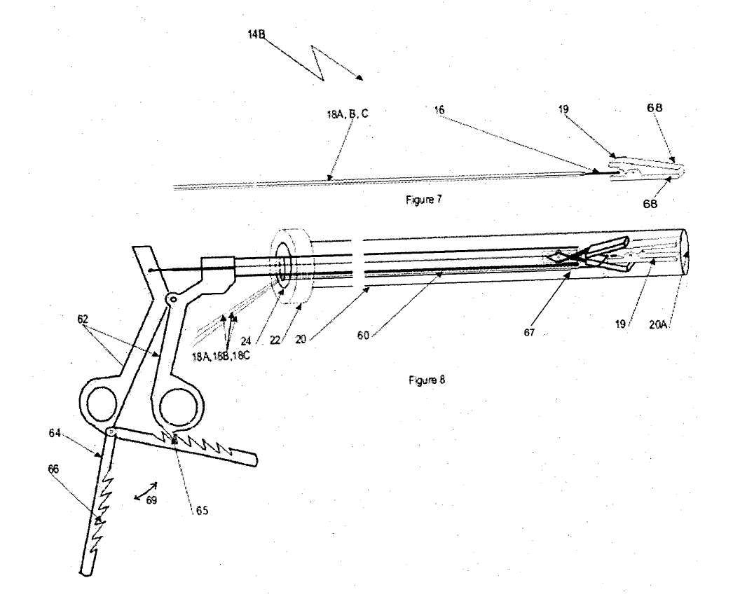

FIG. 5 illustrates a hook anchor 14A, which is to be inserted into the introducing sheath 20 by holding it with a grasper assembly 60 (see FIG. 6). The hook Anchor 14A has a shank 17S ending with one or more flukes 17 formed on the crown on one side, the fluke/s gently curve backwards. This shank 17S is attached to a common string 16 provided on the other side, which is subsequently divided into multiple flexible strings 18A, 18B and 18C.

FIG. 6 illustrates the hook Anchor 14A described above is introduced by holding it with a conventional grasper assembly 60 partially inserted through the opening 24A provided in the washer 24 snugly fitted on the annular disc 22, which in turn is fitted at the outer end 20B of the introducer sheath 20. The flexible strings 18A, 18B and 18C attached via common string 16 to the hook anchor 14A traverse the entire length of the introducing sheath 20 and exit from the outer expanded annular disc 22 of the introducing sheath 20 through the opening 24A. The ends of flukes 17 can be sharp enough for easy penetration of the tissue of interest; however they can also be made blunt for the tissues, which are relatively softer. In addition, there is a short thread 17A attached to the crown of the hook anchor 14A, which helps in its removal at the end of the surgery. The shank 17S of the hook Anchor 14A is held with the grasping jaws 67 of the grasper assembly 60 having a pawl and ratchet arrangement. This pawl and ratchet arrangement allows the surgeon to grasp or release the shank 17S of the-hook Anchor 14A. The grasper 60 has handles 62 with a pawl and ratchet type arrangement, having two holding arms 62, one of which is provided with a pawl 65 engagable by a ratchet 66 configured on the pivoting ratchet arm 64 provided on the other holding arm 62. The two headed arrow 69 shows the direction of movement of the ratchet arm 64 for engaging or disengaging with the pawl 65. The hook anchor 14A is a useful device to obtain an uninterrupted view of the region around esophageal hiatus. The flukes 17 of the anchor 14A can be engaged in the diaphragm in an area anterior to the esophageal hiatus. The flexible strings 18A, 18B and 18C exit from the upper abdominal wall in such a way that the left lobe of liver remains retracted and thus exposes the area around esophageal hiatus. At the end of the surgery, the flukes 17 are disengaged, strings 18A, 18B and 18C are cut either inside or outside of the abdominal wall and hook anchor 14A is pulled into the introducing sheath 20 by holding the thread 17A attached to the hook anchor 14A by using the grasper assembly 60 and subsequently removed from the abdomen. The skin guards 70 and pivotable clip 80 provided with strings 18A, 18B and 18C are not shown here for sake of brevity.

FIG. 7 illustrates a different type of anchor, i.e. a clip anchor 14B attached with a common string 16, which is also subsequently divided into flexible strings 18A, 18B and 18C.

FIG. 8 illustrates the grasper assembly 60 partially inserted through the opening 24A (not shown) of the washer 24 fitted on the annular disc 22, which in turn is fitted at the outer end 20B of the introducer sheath 20. The grasper assembly 60 is shown with its grasping jaws 67 grasping the clip anchor 14B (described above) disposed near the inner end 20A of the introducer sheath 20. The flexible strings 18A, 18B and 18C extending out of the washer 24 are similarly used from outside of the abdomen to manipulate Hartman's pouch HP of the gallbladder or any other internal organ. The construction of the grasper assembly 60 is similar to that described in respect of FIG. 6 above. The clip anchor 14B has two interlocking prongs 68 of plastic, metal or wooden material and there is a small-coiled fulcrum made up of a single wire (not shown). The coiled single wire (not shown) keeps both the prongs 19 held together and by closing the open ends, the closed end can be opened. The grasper assembly 60 holds the prongs 19 at the open end of the clip anchor 14B, so that it remains open. The grasper assembly 60 and the clip anchor 14B attached to the common string 16 along with flexible strings 18A, 18B and 18C are all housed together in the introducing sheath 20. When the clip anchor 14B is ready to grasp Hartman's pouch HP or any other internal organ, then the jaws 67 of the grasper assembly 60 are released which allows the Clip Anchor 14B to hold the internal organ, Then, each of the flexible strings 18A, 18B and 18C are brought out of the abdominal wall individually in the region selected by the surgeon and the internal organ is manipulated using these strings in the required direction to expose the area of interest, as to be described subsequently. The skin guards 70 and pivotable clip 80 provided with strings 18A, 18B and 18C are not shown here for sake of brevity.

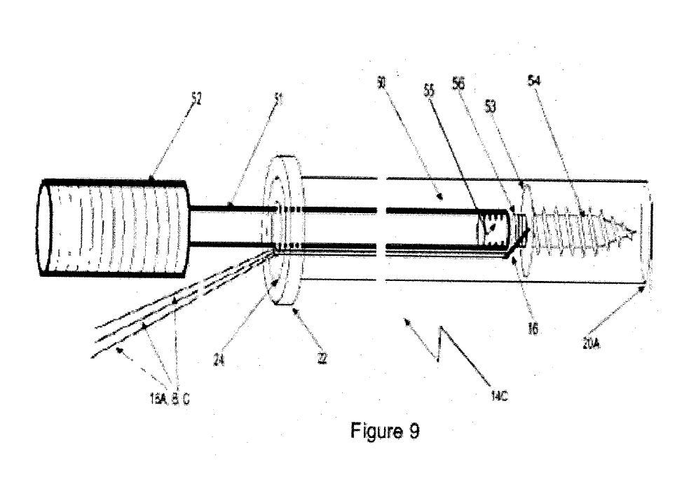

FIG. 9 illustrates another type of anchor, i.e. a screw anchor 14C along with the screwdriver 50. The screw anchor 14C having a screw head 54, a non flexible annular disc 53, and external helical threads at the rare end 56 is disposed near the inner end 20A of the introducer sheath 20. The screwdriver 50 has a cylindrical arm 51 and handle 52 at its outer end for manipulating the screw anchor 14C from outside of the introducer sheath 20. A non-flexible annular disc 53 is provided at the rear end of the screw head 54 so that only the head portion of the screw anchor 14C up to annular disc 53 advances in fibroid of the uterus or any other benign tumor. The screw anchor 14C is also provided with external helical threads 56 at its rear end for screwing the cylindrical arm 51 of the screwdriver 50, which is provided with complementary internal threads 55. The figure shows the screwdriver 50 and screw anchor 14C in an unscrewed position. In addition, the screw head 54 has a plurality of spikes in the anti-clockwise direction to prevent its detachment from the fibroid of the uterus or benign tumor in the abdomen, once the screw anchor 14C is positioned therein. The common string 16 of screw anchor 14C is attached to the non-flexible annular disc 53, which is subsequently divided into flexible strings 18A, 18B and 18C exiting from the opening 24A (not shown) of the washer 24 fitted over the annular disc 22 of the introducer sheath 20, as already described earlier. These strings 18A, 18B and 18C are also used in a similar manner from outside of the abdomen to manipulate the fibroid of the uterus or solid benign tumor in the abdomen. The skin guards 70 and pivotable clip 80 provided with strings 18A, 18B and 18C are not shown here for sake of brevity.

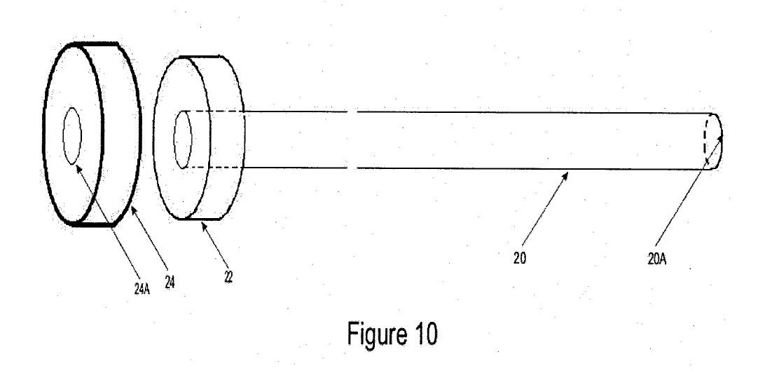

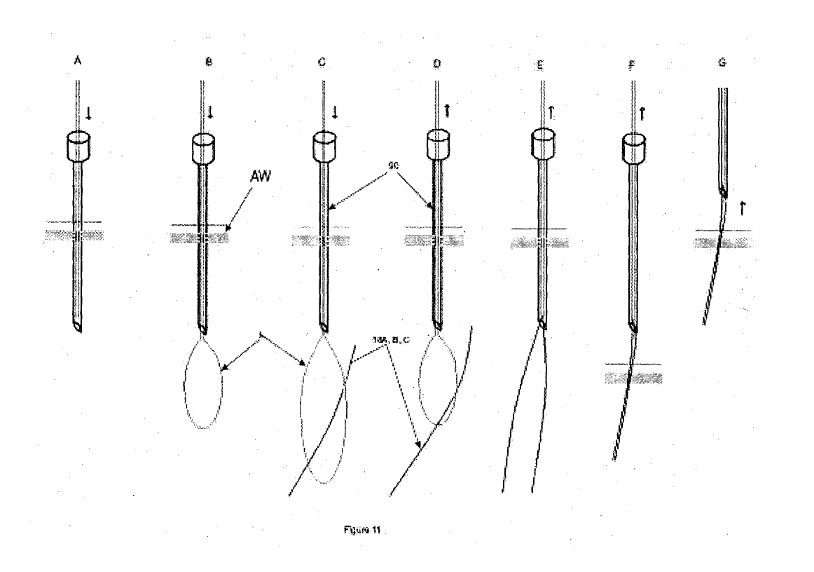

FIG. 10 illustrates the introducer sheath 20 with an embodiment of the annular disc 22 disposed at its outer end 20B. A washer 24 with an opening 24A is also provided for covering this disc 22.

The introducer arrangement will vary from type of anchor to be used. In the case of the T-fastener Anchor 14, it is configured as the slotted introducer needle 30 with T-fastener pusher 40; in the case of a hook anchor 14A and clip anchor 14B, it is configured as a grasper assembly 60 and in case of a screw anchor 14C, it is configured as a screwdriver 50. The introducer sheath 20 in turn is introduced through a trocar during the laparoscopic surgery. A washer 24 is provided with one central opening 24A for inserting the slotted introducer needle 30, grasper assembly 60 or screwdriver 50. The outer end 20B of the introducer sheath 20 is configured as an annular disc 22, and the washer 24 is snugly fitting over it.

FIG. 11 illustrates a long conventional hypodermic needle 90 used for bringing the strings 18A, 18B and 18C from inside to the outside of the abdominal wall AW. Hypodermic needle 90 is a regular needle, which has a hollow tubular structure. Its inner end is sharp, so that it can easily pierce through the abdominal wall. Its outer end expands into a short hollow cylinder, so that the needle 90 can be handled easily. The hypodermic needle 90 with a suture loop L is passed through it, pulls each of the flexible strings 18A, 18B and 18C from inside to the outside of the abdomen. This is done by inserting the hypodermic needle 90 at the selected site on the abdominal wall AW, from where the flexible strings 18A, 18B and 18C needs to be pulled out. The sharp end of the needle 90 is seen laparoscopically and then the suture loop L is pushed into the hypodermic needle 90 (Step A), which in turn takes its shape within the abdominal cavity (Step B). One of the flexible strings, for e.g., 18A of the anchor is guided through this suture loop L (Step C) and then the suture is pulled through the external end of the hypodermic needle 90 (Step D). By doing this, the string 18A is caught into the suture loop L (Step E). Then, the hypodermic needle 90 is pulled out of the abdominal wall AW, which in turn pulls out the string 18A of the anchor (Step F). Then, the suture thread loop L is pushed again into the hypodermic needle 90 releasing the string 18A and then the loop L of the suture is taken to the second exit site to pull the second string 18B and then 18C. Around each of the exit sites of these strings 18A, 18B and 18C, a respective skin guard 70 configured with a string director 78 is also placed. In each steps A through F, the respective arrows show the direction of movement of the suture loop L through the needle 90.

FIG. 12 illustrates views A, B, C of the skin guard 70 configured with a string director 78 for directing flexible strings 18A, 18B and 18C of the laparoscopic device in accordance with the present invention, which is attached to a respective anchor as discussed above. The skin guard 70 is provided to protect the skin by reducing friction at the site of exit site of the respective strings 18A, 18B and 18C from the abdomen and to retain it on the skin guard 70 in order to direct it towards the pivotable clip 80. The front view A of the skin guard 70 shows a slit 72 and the base 76 thereof. The side view B shows the position and configuration of the string director 78 configured and the inclined slope 74 for supporting the respective flexible strings 18A, 18B and 18C thereon. The top view C clearly shows the specific configuration of the slit 72 and the string director 78 in its specific profile. The skin guard 70 is generally cylindrical in shape. The string director 78 is formed by indenting the top face of the skin guard 70 by two to three mm. The lower end of the slit 72 and this indented top face joined by a sloped portion 74. This slop 74 supports and directs the respective string 18A, 18B and 18C towards the indented top face of the string director 78 and then towards the pivotable clip 80 to fix them therein. The bottom face 76 of the skin guard 70 has an adhesive surface like a sticker, which can be easily exposed by peeling away the protective paper sticking thereon. This adhesive surface helps to fix the skin guard 70 at the desired location. The indented top portion of the string director 78 also helps in steadying the respective string 18A, 18B and 18C.

FIG. 13 illustrates the pivotable clip 80 of the laparoscopic device in accordance with the present invention, which is used to fix the respective strings 18A, 18B and 18C on the abdominal wall. The surgeon from outside of the abdomen manipulates these strings during the laparoscopic surgery in order to move the internal organ. The pivotable clip 80 is rectangular in shape. The pivotable clip 80 consists of two rectangular parts, an upper part 82 and a lower part 84. The strings 18A, 18B and 18C are caught between these two rectangular parts 82 and 84. The sides facing each other of the upper and lower parts are serrated for better gripping of the strings 18A, 18B and 18C. These two parts 82 and 84 are connected to each other by a known pivot arrangement on one side (not shown), about which the upper part 84 can be swiveled with respect to the lower part 82. The other side has a conventional locking system (also not shown), by which both these parts 82 and 84 can be locked to each other. By locking these parts 82 and 84 of the pivotable clip 80, the strings 18A, 18B and 18C are fixed at two points, i.e. inside the abdomen to the internal organ and outside the abdomen in the pivotable clip 80. As each of the string 18A, 18B and 18C are now fixed; the internal organ can be easily manipulated by the surgeon in the required direction by pulling any of these strings. The pivotable clip 80 is fixed at the selected area on the abdominal wall in such a way that the surgeon can ergonomically move each of these strings 18A, 18B and 18C with the shoulder joints in natural position. This reduces strain to the shoulder joints of the surgeon. The base of this pivotable clip 80 also has an adhesive surface, which can be exposed by peeling-of the protective paper sticking thereon.

FIG. 14 illustrates the cross-sectional view of the abdomen showing an operative region, e.g. abdominal cavity AC, Hartman's pouch HP of the gallbladder GB that is to be manipulated by means of a laparoscopic device in accordance with the present invention. The flexible strings 18A, 18B and 18C are shown coming out of the abdomen duly supported on the indented top portion of the string director 78 of the skin guard 70. A T-fastener 12 (according to the present inventor's earlier patent application No. 3012/MUM/2012) is also inserted into the fundus of the gallbladder (FGB) to keep it pulled towards the thoracic diaphragm.

The description provided herein is purely by way of example and illustration. The various features and advantageous details are explained with reference to the non-limiting embodiments in the following description of the present invention. The descriptions of well-known components and processing techniques are consciously omitted so that the embodiments described in this specification are not unnecessarily obscured.

The exemplary embodiments described in this specification are intended merely to provide an understanding of the various manners in which these embodiments may be used and to further enable the skilled person in the relevant art to practice these embodiments. Accordingly, these illustrative exemplary embodiments should not be construed as limiting the scope of the present invention.

It is to be understood that the phraseology or terminology employed in this specification is merely for the purpose of description and not for limiting the scope thereof. Therefore, although the embodiments in this disclosure have been described in terms of its preferred embodiments, the skilled person in the relevant art would readily recognize that these embodiments can be applied with modifications possible within the spirit and scope of the present invention as described in this specification.

### Working Principle of the Laparoscopic Device

The patient is kept under general anesthesia. For a standard laparoscopic cholecystectomy, the umbilical trocar is inserted and preliminary inspection is carried out. Subsequently, another trocar is inserted midway between the costal margin and the umbilicus in midclavicular line. Then, the telescope is shifted to the midclavicular trocar and laparoscopic device with a T-fastener as described in the Indian patent application no. 3012/MUM/2012 is used for anchoring the fundus of the gallbladder FGB with the diaphragm. Now, the Laparoscopic device in accordance with the present invention having a anchor 14 is selected, which is attached to a common string 16 subsequently divided into multiple flexible strings 18A, 18B and 18C is introduced through this umbilical trocar.

In case of single incision laparoscopic surgery, after insertion of the port/ports at the umbilicus, the laparoscopic device in accordance with this invention is introduced inside the abdomen guided by the laparoscopic vision.

The inner end 20A of the introducer sheath 20 is then brought in contact with Hartman's pouch HP of the gallbladder. The hook 36 locked on the annular disc 22 of the introducer sheath 20 is unlocked and the released introducer needle 30 is pushed into the introducer sheath 20, which in turn pierces Hartman's pouch of the gallbladder. Then, the hook 46 of the T-fastener pusher 40 is unlocked and released T-fastener pusher 40 is pushed inside the slotted introducer needle 30 and by this maneuver, T-fastener 14 is pushed out of the slotted introducer needle 30 and there by delivered into the lumen of the gallbladder. Subsequently, the T-fastener pusher 40 is removed and the outer end 30B of the slotted introducer needle 30 is connected to a suction apparatus. The bile from the gallbladder is sucked out to decompress the gallbladder for its subsequent handling with ease. In addition, this would also reduce the spillage of the bile in the operating area. This prevention of spillage is further enhanced by the annular sealing ring 17B, which is present at the junction of the common string 16 with T-fastener 14. This sealing ring 17B shall effectively close the opening in the gallbladder from the inside. The slotted introducer needle 30 is removed after completion of suction of the bile from the gallbladder. If the bile in the gallbladder is thick, then repeated actions of suction and irrigation could be performed to aspirate as much of it as possible.

The next step is to transfer the flexible strings 18A, 18B and 18C (attached via common string 16 to T-fastener 14) from the inside to the outside of the abdomen. Before starting this step, the skin guard and pivotable clip is separated from the strings. Then, the first string 18A is pulled from the epigastric region. This is done by means of a long conventional hypodermic needle 90 inserted with a suture loop L is introduced through the abdominal wall at the selected site on the abdominal wall. The inner end of this needle 90 is then seen laparoscopically and the suture is pushed into the needle and which in turn is inserted into the abdomen to take the shape of loop L. The end of the first string 18A is held by the grasper assembly 60 at the outer end 20B of the introducing sheath 20 and grasper assembly 60 is pushed into the introducer sheath 20 and then into the abdomen. Subsequently, the end of the string 18A is passed through this suture loop L. Now, the grasper assembly 60 is withdrawn and so the suture loop L is pulled into the needle 90, this maneuver will catch the string 18A into the suture loop L. Subsequently, the needle 90 along with the suture loop L engaging the first string 18A is pulled out of the abdomen. Finally, the first string 18A caught in the suture loop L is freed and pulled out of it by again pushing the suture into the needle 90.

This procedure is similarly repeated to pull out the second string 18B just under the costal margin in the midclavicular area. Similarly, the third string 18C is pulled out under costal margin in the anterior axillary line. Subsequently, the adhesive surface of the skin guard 70 is exposed by peeling away the paper sticking at its bottom face 76. The skin guard 70 is now fixed to the skin at the respective exit sites of each of the strings 18A, 18B and 18C to protect the skin and to direct these strings 18A, 18B and 18C towards the pivotable clip 80 in their respective fixed positions thereon. Now, the adhesive surface of the pivotable clip 80 is also exposed by peeling away the paper sticking at its bottom face and the pivotable clip 80 is also fixed to the selected area over the abdominal wall, such that each of the strings 18A, 18B and 18C can be pulled easily without causing any strain to the shoulder joints of the surgeon.

Operating surgeon controls these strings 18A, 18B and 18C like chords of the guitar. The location and color of the flexible strings 18A, 18B and 18C in the pivotable clip 80 helps the surgeon to easily recognize which string needs to be pulled, depending upon the requirement of the laparoscopic dissection.

For the purpose of easier understanding, a pull on the first string 18A which is shown on the left side in the pivotable clip 80 in FIG. 13 will expose the lateral part of Hartman's pouch and Calot's triangle. A pull of the right string 18C will expose the medial part of Hartman's pouch and Calot's triangle. A pull on the central string 18B will expose the central area.

The combination of these strings 18A, 18B and 18C can be pulled for the rest of the dissection. By using this technique, the cystic artery and duct is dissected in the Calot's triangle. The rest of the procedure is completed as usual. After completion of the separation of the gallbladder from the liver fossa, these flexible strings 18A, 18B and 18C are cut near the common string 16. The T-fastener anchor remains in the gallbladder and is removed along with common string 16 with the specimen of the gallbladder. The divided strings 18A, 18B and 18C are removed by pulling them from the exit site on the abdominal wall AW. Then, the skin guard with string director 70 and pivotable clip 80 are separated from the skin and removed.

The Laparoscopic device having “hook anchor 14A attached with a common string 16 subsequently divided into flexible strings 18A, 18B and 18C” is useful for the retraction of the liver. To carry out this maneuver, the introducing sheath 20 is brought near the area anterior to the esophageal hiatus on the diaphragm. The grasper assembly 60 holding the-hook anchor 14A is pushed out of the introducing sheath 20 and under the laparoscopic vision, the flukes 17 of the hook anchor 14A are fixed in the diaphragm anterior to the esophageal hiatus. The flexible strings 18A, 18B and 18C are brought out from the selected area and fixed on the abdominal wall as described earlier for the T-fastener anchor 14. This maneuver will retract the left lobe of the liver effectively. It will allow an easy access to the esophageal hiatus in order to carry out the rest of the surgical steps, in surgeries, such as-fundoplication and esophagocardiomyotomy. At the end of the procedure, the fluke/s 17 are disengaged from the diaphragm and hook anchor 14A is pulled into the introducing sheath 20 using the thread 17A attached to the crown of the hook anchor 14A and the hook anchor 14A is removed along with introducer sheath.

The laparoscopic device “Clip Anchor 14B attached with a common string 16 subsequently divided into flexible strings 18A, 18B and 18C” is a useful device in retraction of the hollow viscous body. The introducer sheath 20 is brought near the hollow viscous body, which needs to be manipulated. The grasper assembly 60 holding this clip anchor 14B is pushed out of the introducer sheath 20. The ratchet 65 of the grasper assembly 60 is unlocked and blade of handle 62 is opened which in turn opens the jaws 67 of the grasper 60 allowing it to close the jaws of the clip 19. The jaws of the clip anchor 14B are closed in such a way that it holds the internal organ of interest. The attached strings 18A, 18B and 18C are brought out of the abdomen from the selected area and fixed on the abdominal wall by the same maneuver as described earlier, by using the suture loop L. The external movement of the strings 18A, 18B and 18C will manipulate the internal organ and the dissection is carried out to complete the procedure. At the end of the procedure, the grasper assembly 60 is used to open up the prongs 19 of the clip anchor 14B and it is freed from the internal organ. The strings 18A, 18B and 18C are cut individually and pulled from outside of the abdomen. The common string 16 of the clip Anchor 14B is pulled into the introducer sheath 20 and the clip anchor 14B is removed along with the introducer sheath 20.

Similarly, the “Screw Anchor 14C attached with a common string 16 subsequently divided into flexible strings 18A, 18B and 18C” is a useful laparoscopic device for manipulating the relatively solid tissue, like fibroid of the uterus and solid benign tumors. In these conditions, the laparoscopic device with a screw anchor 14C is used. The introducer sheath 20 of the device is brought in contact with area of interest and the screw anchor 14C is screwed by clockwise rotation using the external handle 52. Once, the entire length of the screw anchor 14C is screwed inside the solid organ or tumor, an anti-clockwise rotation is made. The anticlockwise spikes provided on the screw threads 54 prevents the screw anchor 14C from getting unscrewed, however the screwdriver 50 is detached from the screw anchor 14C and then removed.

The respective flexible strings 18A, 18B and 18C are brought out and fixed on the abdominal wall at selected sites as mentioned earlier. The movement of these strings 18A, 18B and 18C pulls the solid organ in the required direction to complete the dissection and thereby the laparoscopic procedure. Once, the procedure is completed, these strings 18A, 18B and 18C are cut near the screw anchor 14C and are pulled out of the abdomen. The screw head 54 remains in the fibroid of the uterus or solid benign tumor and is subsequently removed along with the specimen.

### Technical Advantages & Economic Significance

It is possible to reduce the number of trocars used in a laparoscopic surgery using the present invention and thus reducing the cost of the surgery.

The use of present invention offers the laparoscopic surgeon an ergonomically superior position, specifically to reduce the abduction at the shoulder joints and thus reducing the associated wear and tear of shoulder joint.

By use of this invention it is possible to reduce the number of instruments to be inserted from the umbilicus during a single incision laparoscopic surgery.

By use of this laparoscopic device, there is increase in the freedom of movement of the instruments, which are inserted through the umbilicus, and in addition it facilitates the ease with which single incision laparoscopic surgery can be carried out by the laparoscopic surgeon.

The use of the present invention simplifies the single incision laparoscopic cholecystectomy by enabling a good retraction of the Hartman's pouch of the gallbladder and satisfactory exposure of Calot's triangle, allowing safe dissection of Calot's triangle, thereby aiding to reduce the chances of injury to the bile ducts (tubes that carry bile from the liver to the intestine).

By use of present laparoscopic device, it is possible to dispense with the myoma screw and an assistant in laparoscopic excision of fibroid of the uterus.

Use of present invention allows easy manipulation and retraction of the uterine fibroid and benign solid tumors during their laparoscopic surgical excision.

It is possible to have a good continuous retraction of the left lobe of the liver during laparoscopic surgery being performed at the esophageal hiatus by use of the present invention.

### Other Uses of the Present Invention:

The principle of anchor attached to multiple strings can be used in any other endoscopic or open surgery with associated benefits. In addition to achieve the manipulation of the internal tissues, the principle can also be used in suturing of the internal tissues.

Any discussion of documents, acts, materials, devices, articles or the like that has been included in this specification is solely for the purpose of providing a context for the invention. It is not to be taken as an admission that any or all of these matters form part of the prior art base or were common general knowledge in the field relevant to the invention as it existed anywhere before the priority date of this application.

While considerable emphasis has been placed herein on the specific features of the preferred embodiment, it will be appreciated that many additional features can be added and that many changes can be made in the preferred embodiments without departing from the principles of the invention. These and other changes in the preferred embodiment of the invention will be apparent to those skilled in the art from the disclosure herein, whereby it is to be distinctly understood that the foregoing descriptive matter is to be interpreted merely as illustrative of the invention and not as a limitation.

I claim:

1. A laparoscopic device configured with an ergonomic external control to manipulate an internal organ, the laparoscopic device comprises:
an introducer sheath having an open inner end and an open outer end fitted with an annular disc;
an opening in the annular disc that is adapted to be closed in a sealing manner by means of a washer;
at least one anchor attached to a common flexible string, which is subsequently divided into a plurality of flexible strings extending out of the introducer sheath through the opening in the washer; the anchor being disposed inside the introducer sheath;
means for securing the anchor inside the introducer sheath and for releasing the anchor into the abdomen;
wherein the plurality of flexible strings passing through the opening in the washer are adapted to be fixed in a pivotable clip and each of said plurality of strings passes through a skin guard having a string director for manipulating the internal organ with ergonomic external control.

2. Device as claimed in claim 1, wherein the anchor is configured as at least partially magnetic T-fastener, which also has a substantially annular flexible disc disposed at the junction of the common flexible string with the T-fastener.

3. Device as claimed in claim 2, wherein the means for introducing the T-fastener comprises of an introducer needle having an open, sharp, slotted inner end and an open outer end provided with an annular disc having a locking arrangement.

4. Device as claimed in claim 3, wherein the locking arrangement comprises of a pivoting arm extending from the annular disc of the slotted introducer needle and having a hook lockable on the annular disc of the introducer sheath to lock the introducer needle in the introducer sheath or to release the introducer needle from the annular disc of the introducer sheath to move the introducer needle out of the introducer sheath.

5. Device as claimed in claims 2 to 4, wherein means for securing the T-fastener is a magnet fitted adjacent the slotted end of the introducer needle.

6. Device as claimed in claims 2 to 5, wherein means for releasing the T-fastener out of the introducer needle is T-fastener pusher provided with a circular disc having a locking arrangement which is lockable on the annular disc of the introducer needle to lock the T-fastener pusher inside the introducer needle and to release the T-fastener pusher in order to push the T-fastener out of the introducer needle and thereby to release T-fastener into the internal organ.

7. Device as claimed in claim 6, wherein the T-fastener pusher has a concaved profile at its end abutting the T-fastener and the T-fastener has complementary convex profiles at both ends thereof.

8. Device as claimed in claim 1, wherein the anchor is configured as a hook anchor having at least one fluke.

9. Device as claimed in claim 1, wherein the anchor is configured as a clip anchor.

10. Device as claimed in claim 8 or 9, wherein the means for securing the hook anchor or clip anchor inside the introducer sheath is a grasper assembly.

11. Device as claimed in claim 10, wherein the grasper assembly comprises of the locking means partially disposed outside the introducer sheath and the grasping means disposed inside the introducer sheath, the locking means and grasping means cooperating with each other to grasp or release the hook anchor or clip anchor.

12. Device as claimed in claim 8, wherein the anchor comprises of a hook anchor having at least one fluke and shank of the hook anchor is attached with a common flexible string on one side and a piece of thread attached to the opposite side.

13. Device as claimed in claim 1, wherein the anchor is a screw anchor.

14. Device as claimed in claim 13, wherein the screw anchor is secured and released by a screwdriver having a cylindrical arm provided with internal threads at its front end for screwing or unscrewing complementary external threads of screw anchor and a handle firmly fitted at its rear end which is disposed outside the introducer sheath.

15. Device as claimed in claims 13 to 14, wherein the common flexible string of the screw anchor is attached to a non-flexible disc fitted at the rear end of the screw head, the non-flexible disc acts as a stopper while the screw anchor is being screwed into an internal organ, the external screw threads of the screw anchor provided at its rear end are adapted to be engaged by the internal threads of the handle of the screwdriver.

16. Device as claimed in claims 14 to 15, wherein the screw head is provided with a plurality of spikes in the anti-clockwise direction for preventing detachment of the screw head from the internal organ.

17. Device as claimed in any of the preceding claims, wherein the pivotable clip comprises of an upper and lower rectangular portion which are adapted to be pivoted with respect to each other on one side thereof and also adapted to be locked to each other on the other side thereof for fixing the respective string in position within the pivotable clip.

18. Device as claimed in any of the preceding claims, wherein the pivotable clip comprises of a base provided with an adhesive surface having a removable cover for sticking it on the abdomen.

19. Device as claimed in any of the preceding claims, wherein the skin guard is configured with an inclined slope to protect the skin by reducing friction at the exit site of the respective string and to retain the string on the skin guard and to direct it towards the pivotable clip.

20. Device as claimed in any of the preceding claims, wherein the skin guard is configured as a cylindrical body with a through slit at one point on the circumference thereof, the slit extending proximate to the central region of the skin guard and then transforming into a smooth inclined slope ending near its top face which is depressed by a predetermined height to configure a depression from the top surface of the skin guard thereby forming a grooved recess to retain the string on the skin guard.

21. Device as claimed in any of the preceding claims, wherein the skin guard comprises of a base, which is provided with an adhesive surface having a removable cover for sticking it on the abdomen.

22. Device as claimed in claim 1, wherein the common flexible string comprises a plurality of multiple flexible strings corresponding to each of the multiple flexible strings.
